(19) United States
(12) Patent Application Publication
(10) Pub. No.: US 2009/0210035 A1
(43) Pub. Date: Aug. 20, 2009

(54) System for powering medical implants

(76) Inventor: Daniel Gelbart, Vancouver (CA)

Correspondence Address: DANIEL GELBART, 4706 DRUMMOND DR, VANCOUVER, BC, V6T-184, CA

(21) Appl. No.: 12/071,317

(22) Filed: Feb. 20, 2008

(51) Int. Cl. A61N 1/378

(52) U.S. Cl. 607/61

(57) ABSTRACT

A medical implant is powered by inductive coupling to a transmitter utilizing a large number of frequencies in order to minimize the amount of electromagnetic interference at any single frequency. The frequencies are generated by a resonant circuit rapidly tunable by changing the inductance.

### FIELD OF THE INVENTION

The invention relates to the medical field and in particular to powering medical implants by wireless coupling of energy.

### BACKGROUND OF THE INVENTION

Many medical implants require electrical power. In general there are four ways to supply the electrical power: primary batteries, rechargeable batteries, electromagnetic coupling and bio-electric sources. The best known example of an electrical implant is a cardiac pacemaker. Most of the volume of the pacemaker is taken up by the primary batteries, which need to last for several years. To decrease battery size or eliminate the battery altogether, wireless transmission of power can be used. A coil inside the implant picks up the alternating current (AC) magnetic filed of a larger coil located outside the body. The output of the coil is rectified and can be use to power the implant or charge up the batteries of the implant. Well known examples of such systems are cochlear implants (with rechargeable batteries) or microstimulators such as the BION, having no batteries and relying on the continuous transmission of power. Other implants can be used for sensing and monitoring. Some implants are used for identification, as the well known RFID. Most of those systems operate in the frequency range of 100 KHz to 1 MHz but operation at high frequencies such as 50 MHz is easily possible, as the human tissue attenuating of electromagnetic waves only increases rapidly at higher frequencies. A typical prior art system is shown in FIG. 1. An implant 1 located inside tissue 7 is powered by inductive coupling to a transmitter 6 having a coil 8. Inside the implant the AC signal from coil 2 is rectified by rectifier 3 and powers the electronics 4. In case of a stimulator, the output pulses are transmitted to the body by electrode 5. Since most of the radiated power does not reach the implant, it is desired to increase the magnetic field strength of coil 8 without requiring more power. This is done by resonating coil 8 with capacitor 9 and powering it by oscillator 10. This well known technique increases the current in the coil by a factor of up to a thousand times. The main limit on the transmitted power of all these systems is the fact they radiate electromagnetic interference (EMI) that can interfere with the operation of other equipment. In most countries EMI is regulated; for example in the US it is regulated by the Federal Communications Commission (FCC). The standard that sets the EMI limits for “intentional radiators” (the term used by the FCC for such systems) is known as FCC Part 15. A typical FCC EMI test is shown in FIG. 2. Graph 12 is the FCC EMI limit and graph 11 is the EMI spectrum of the transmitter. As expected, there is a sharp spike 13 at the transmitted frequency. When the amplitude of 13 exceeds graph 12 the device can not be sold or used. It is an object of the invention to transmit a large amount of power without creating a large amount of EMI at any given frequency. Any transmitter, by definition, will create EMI. The invention allows spreading the EMI over many frequencies without having any large EMI at any given frequency. Another object of the invention is the use of a resonant circuit for the transmitter, in order to use a small amount of input power to create strong AC magnetic or electric field. The idea to spread the transmitted power over many frequencies is known as “Spread Spectrum”, but it normally precludes the use of a resonant circuit.

### SUMMARY OF THE INVENTION

A medical implant is powered by inductive coupling to a transmitter utilizing a large number of frequencies in order to minimize the amount of electromagnetic interference at any single frequency. The frequencies are generated by a resonant circuit rapidly tunable by changing the inductance.

### BRIEF DESCRIPTION OF THE DRAWINGS

FIG. 1 is a simplified electrical schematic of the prior art.

FIG. 2 is an electrical spectrum graph of a prior art transmitter.

FIG. 3 is an electrical schematic of a tunable resonant circuit according to the invention.

FIG. 4 is a graph of the magnetic properties of the ferrite core used in the resonant circuit.

FIG. 5 is an electrical spectrum graph of a transmitter using the invention.

FIG. 6 is an electrical schematic of a tunable resonant circuit according to the invention using switched capacitors.

### DETAILED SPECIFICATION

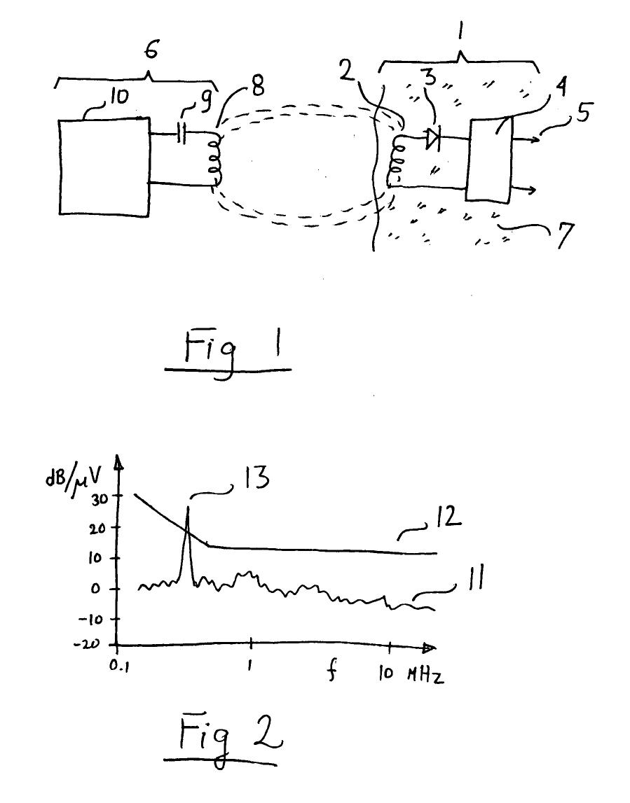

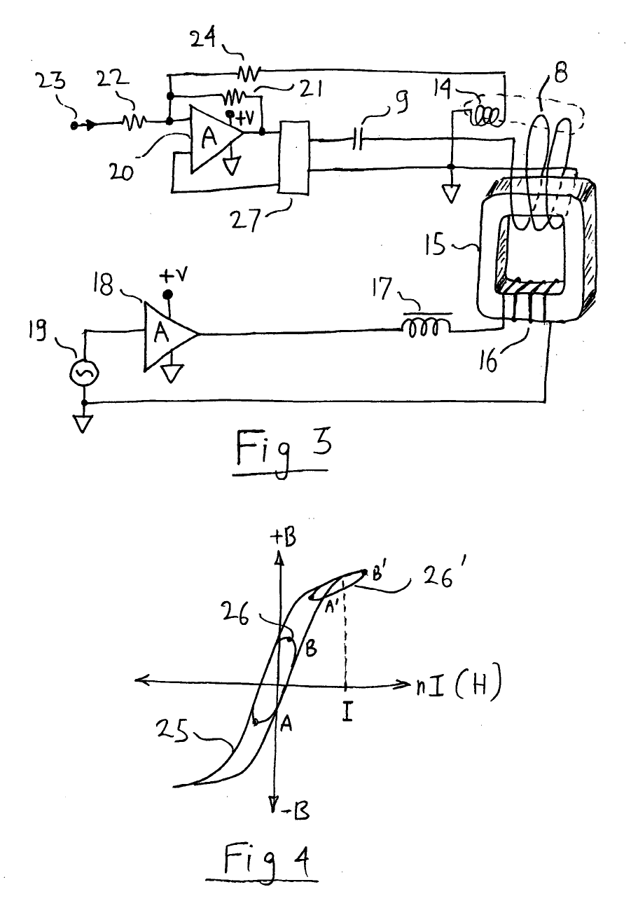

The invention spreads the transmitted energy over many frequencies to minimize the amount of electromagnetic interference at any single frequency. Since the receiver coil (coil 2 in FIG. 1) is not tuned to any specific frequency, spreading the energy over many frequencies does not interfere with the operation of the receiver. Even when coil 2 is tuned by a capacitor the bandwidth of the heavily loaded circuit will allow the use of a wide frequency range. The preferred embodiment of the transmitter uses fast frequency sweeping in order to spread the transmitted power. This allows the benefits of a resonant circuit and minimizes the cost and input power to the transmitter. When a coil and a capacitor form a resonant circuit the current in the coil can be increased many folds over the current with no resonance. The amount of increase is sometimes referred to as the Q of the coil. Typical Q of resonant circuits is between 5 and 500. The higher the Q the narrower the frequency range the circuit can respond to. For example, if a resonant circuit operating at 400 KHz has a Q of 100, it can be operated approximately over a frequency range of 400:100=4 KHz. If the spectrum has to be spread over 100 KHz in order to comply with FCC regulations and no tuning is possible, the Q has to be reduced to 4 requiring a 25 fold increase in the drive voltage. Clearly it would be desired to keep the high Q of the circuit while sweeping over a large bandwidth. Some high frequency circuits are tuned by voltage controlled capacitors known as varactors, but the high voltages required in transmitter circuits make it difficult to use them. According to the invention an oscillator using a coil and a capacitor (LC oscillator) is tuned rapidly by changing the inductance of the coil. An alternate embodiment electronically switches capacitors in order to change the resonant frequency in discrete steps. While the invention can be practiced without a resonant circuit, simply by sweeping a frequency or using multiple frequencies, the greatest benefit will be found by using a resonant circuit. Referring now to FIG. 3, a transmitter coil 8 is resonated with capacitor 9 and driven by amplifier 20. Amplifier 20 can be a linear amplifier, but it is desirable to use a switching power amplifier and particularly a MOS-FET based switching amplifier. The technology of switching amplifiers is well known in the art and needs no further details. An optional filter 27 isolates the amplifier from the coil. This is particularly important for EMI reduction when using switching amplifiers. By the way of example, filter 27 can be a C-L-C section. A pick up coil 14 is inductively coupled to coil 8 and provides positive feedback to amplifier 20 via resistor 24 to form a free-running oscillator having a frequency of f=½π(LC)^{1/2}. An amplitude stabilization circuit, also known as AGC, can be added. Resistor 21 provides the correct DC bias. Resistor 23 is used to modulate the output in order to transmit data to the implant. The art of communicating with an implant is well known and used in pacemakers. Coil 8 also is magnetically coupled to ferrite core 15 having a secondary winding 16 and an AC blocking inductor 17. The magnetization inside core 15 can be changed rapidly by changing the current in secondary winding 16, which is powered by amplifier 18 and frequency source 19. Source 19 is typically a sinusoidal oscillator. Amplifier 18 can be a linear or a switching amplifier. The inductance of coil 8 will depend on the magnetic state of core 15, as will become apparent when viewing FIG. 4 in conjunction with FIG. 3.

The inductance of a coil is defined as L=n.dΦ/dI, where n=number of turns, Φ=magnetic flux, I=current. As seen from the magnetization curve 25 of ferrite 15, the ratio dΦ/dI, which is proportional to dB/dH, changes when core 15 is magnetized by coil 16. When current through coil 16 is off and coil 8 oscillates, the magnetic field in the ferrite changes as shown by minor loop 26, with ends A and B. When a DC current is present in coil 16, the oscillations in coil 8 will create a minor loop 26′ with ends at A′ and B′. Because the slope dB/dH of loop 26′ is lower than loop 26, the inductance of coil 8 will be lower as well. By sweeping the inductance between loop 26 and loop 26′ at a frequency controlled by oscillator 19, the resonant circuit formed by coil 8 and capacitor 9 will oscillate at different frequencies but keep a high Q at all frequencies. The rate of sweeping is limited by the Q of the primary LC circuit, comprising of coil 8 and capacitor 9. For best results the frequency of oscillator 19 should not exceed the frequency of the primary LC circuit divided by the Q. By the way of example, if the primary LC circuit oscillates between 350 KHz and 450 KHz at a Q of 100, it would take over 100 cycles to build up full amplitude. If the sweeping circuit sweeps faster than a few KHz full amplitude will not be built up in the primary circuit.

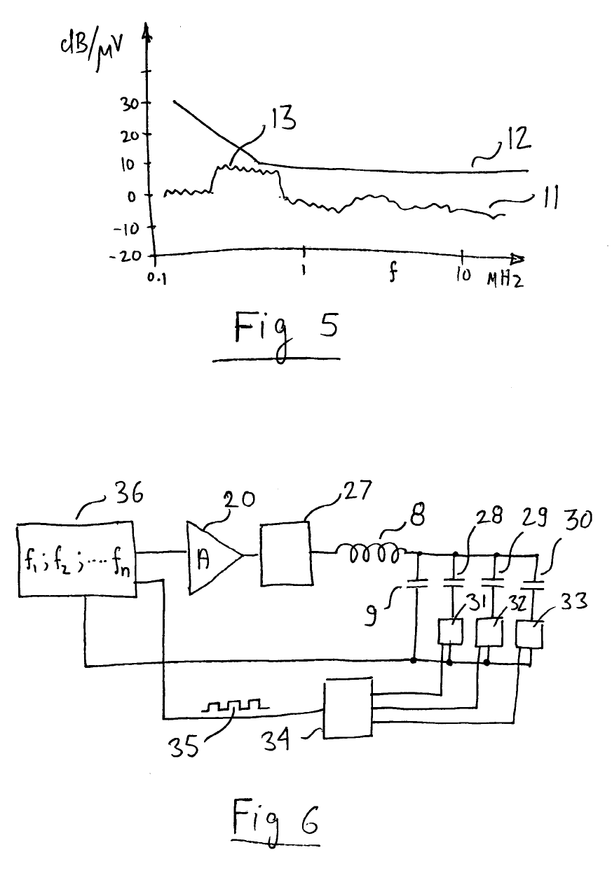

By the way of example, referring to FIG. 3, coil 8 comprises of 20 turns of #12 Litz wire wound on a 300 mm diameter plastic former with a small air gap between the turns. The total inductance in air is about 150 uH but when core 15 is added inductance increases by more than tenfold. Capacitor 9 is a polystyrene capacitor selected to resonate with coil 8 at about 350 KHz. For high Q coils the voltage on capacitor 9 and coil 8 can be many thousands of volts, so the standard high voltage considerations apply. Core 15 and the core of inductor 17 are a ferrite toroid (Fair-Rite Inc part number 5978015901. Multiple cores can be stacked in parallel when larger currents are used in coil 8, to avoid magnetic saturation. Coil 16 has 50 turns of #16 magnet wire and inductor 18 has 1000 turns o same wire. Coil 14 is a small air core coil, about 20 turns on a 50 mm diameter former, placed adjacent to coil 8. All components except the amplifiers are encapsulated with Styrofoam in a plastic enclosure. Amplifiers 18, 29 and filter 27 are mounted in a grounded metal box for shielding. The resonant frequency of the LC circuit was changed from 350 KHz to 450 KHz when a current of a few amps was used in coil 16. Amplifier 18 was a standard switching regulator operated in constant current mode and amplifier 20 was a MOS-FET power amplifier ( Model Ultra 2020 from T&C Power conversion). The sweeping rate of oscillator 19 was about 1 KHz. Resistor 21 was not used and the AGC function of the amplifier was used. When the emission spectrum was observed using a spectrum analyzer, the spectrum appears as shown in FIG. 5. The spectral peak 13 was spread out and stayed below graph 12. In comparison, when the frequency sweeping was stopped, the spectrum appeared as in FIG. 2.

A different embodiment of the same principle is shown in FIG. 6. Instead of a free-running oscillator, a digitally synthesized source 36 produces a discrete set of frequencies. A coil 8 is resonated with capacitors 9, 28, 29 and 30 to match the frequencies of synthesizer 36. The frequency is changed by electronic switches 31, 32, 33 adding capacitors 28, 29, 30 to capacitor 9. Eight different frequencies can be produced by 2 capacitors if capacitor values are powers of 2 (e.g.: 1, 2, 4, 8 etc). Switches 31, 32 and 33, typically MOS-FETs, need to be able to withstand the high voltages produced by a high Q resonant circuit. A counter 34 is clocked by input 35 to select the appropriate switches. If the resonant frequencies are chosen with a spacing of less than f/Q (where f is the center frequency of the LC circuit), continuous frequency sweeping is possible by replacing the discrete frequencies produced by synthesizer 36 with a continuous frequency sweep.

It will be clear to those familiar with the art of RF engineering that many other implementations are possible, such as selecting taps on coil 8 to change frequencies, using varactors, using individual tuned circuits operated in parallel etc.

1. A system for transmitting power to a medical implant, said system using a plurality of frequencies to transmit said power.

2. A system for transmitting power to a medical implant, said system using a sweeping frequency to transmit said power.

3. A system for transmitting power to a medical implant over multiple frequencies, said system using an electronically tunable resonant circuit to transmit said power.

4. A system as in claim 1 wherein said frequencies are transmitted by a single resonant circuit, said circuit made to resonate at multiple frequencies.

5. A system as in claim 1 wherein said frequencies are transmitted by multiple resonant circuits.

6. A system as in claim 3 wherein said resonant circuit is tuned by electronically changing the inductance of an inductor.

7. A system as in claim 3 wherein said resonant circuit is tuned by electronically changing the inductance of an inductor, said inductance is changed by changing the degree of magnetic saturation of a magnetic material forming part of said inductor.

8. A system as in claim 3 wherein said resonant circuit is tuned by electronically changing the capacitance of a capacitor by electronically switching capacitors into the resonant circuit.

9. A system as in claim 3 wherein said resonant circuit is tuned continuously.

10. A system as in claim 3 wherein said resonant circuit is tuned in discrete steps.

11. A system as in claim 3 wherein said resonant circuit is tuned in a sinusoidal manner at a frequency approximately equal to the center frequency of the resonant circuit divided by the Q of the resonant circuit.
